(19) United States
(12) Patent Application Publication
(10) Pub. No.: US 2026/0166332 A1
(43) Pub. Date: Jun. 18, 2026

(54) DEVICE AND METHOD FOR USING OPTICAL EMITTERS TO TREAT OBSTRUCTIVE SLEEP APNEA

(71) Applicant: Terry Ward, Orlando, FL (US)

(72) Inventor: Terry Ward, Orlando, FL (US)

(21) Appl. No.: 19/534,045

(22) Filed: Feb. 9, 2026

Related U.S. Application Data
(63) Continuation-in-part of application No. 17/171,784, filed on Feb. 9, 2021.
(60) Provisional application No. 63/757,038, filed on Feb. 11, 2025.

(51) Int. Cl. A61N 5/06

(52) U.S. Cl. CPC A61N 5/0616; A61N 2005/0626; A61N 2005/0642; A61N 2005/0652; A61N 2005/0662

(57) ABSTRACT

An Obstructive Sleep Apnea treatment device and method utilizing optical emitters includes a device having an array housing a plurality of optical emitters which are positioned to produce an optical output directed to a recipient, and a controller for instructing an operation of the array. A method of treating obstructive sleep apnea includes utilizing the device having the array to reduce the lipid content of subcutaneous adipocytes in the face and neck area of the recipient using via the optical emissions of the device.

### CROSS-REFERENCE TO RELATED APPLICATIONS

This application is a continuation in part to U.S. application Ser. No. 17/171,784 filed on 2 Sep. 2021 and claims the benefit of U.S. Application Ser. No. 63/757,038 filed on 11 Feb. 2025, the contents of each of which are incorporated herein by reference.

### TECHNICAL FIELD

The present invention relates generally to the field of reducing Obstructive Sleep Apnea, and more particularly, to a device and method for reducing head and neck fat utilizing optical emitters.

### BACKGROUND

The statements in this section merely provide background information related to the present disclosure and may not constitute prior art.

As shown in background FIG. 1, Obstructive Sleep Apnea (“OSA”) is a sleep disorder where the upper airway becomes blocked during sleep, causing repeated interruptions in breathing and temporarily cutting off oxygen supply to the body due to the obstruction, often resulting in disrupted sleep and daytime fatigue.

Apnea-Hypopnea Index (“AHI”) is the primary measure used to determine the severity of OSA, as it calculates the average number of apneas and hypopneas a person experiences per hour of sleep, with higher AHI values indicating more severe OSA. A 2020 study showed a correlation of weight loss and reducing the fat in the tongue and oral structures. MRIs in the study found that a 32% weight loss reduced tongue fat by 48% and reduced the recipient's Apnea-Hypopnea Index by 80%.

While excess weight has long been known to be a risk factor for OSA, an increasing amount of evidence suggests the relationship is reciprocal, because sleep deprivation is associated with decreased leptin (an appetite-suppressing hormone) and increased ghrelin (an appetite-stimulating hormone), which may increase cravings for calorie-dense foods. Additional data indicates that insufficient sleep leads to overeating, obesity, and a decrease in fat-loss during calorie restriction.

For these reasons, there are many known OSA treatment options such as lifestyle changes for weight loss, oral appliances such as tongue stabilizers, medication, and surgery. However, the most prevalent treatment devices are called Continuous Positive Airway Pressure (“CPAP”) machines that deliver air pressure through hoses and a mask worn by the recipient to keep the airways open while sleeping.

Although CPAP machines are useful, many user complaints include the uncomfortable nature of the mask, feeling claustrophobic, skin irritation from the mask, excessive air swallowing which lead to bloating and the noise generated by the machine in operation. As a result, it is not uncommon for users to choose not to use their CPAP machine on a daily basis. Moreover, conservative estimates report that around 40% of individuals who receive a CPAP machine stop using them completely within 90 days from the initial purchase.

Accordingly, it would be beneficial to provide a device and method for using optical emitters to treat obstructive sleep apnea that does not suffer from the above noted drawbacks.

### SUMMARY OF THE INVENTION

The present invention is directed a device and method for treating obstructive sleep apnea. One embodiment of the present invention can include an output array that includes a main body having an array of optical emitters that can produce optical emissions which emanate outward from the front surface of the main body. The device can also include a stand and a controller having a user interface for instructing an operation of the array.

In one embodiment of the present invention, the device can generate a target optical output that is configured to deliver a target optical delivery to treat obstructive sleep apnea in the recipient when the array is positioned at a first distance from the target area of the recipient.

In one embodiment of the present invention, the device can include a wireless transceiver that can send and receive information with an externally located device. The externally located device can include a file server, and the file server can control an operation of the array.

Yet another embodiment of the present invention can include a method of treating obstructive sleep apnea by reducing lipid content of subcutaneous adipocytes in the face and neck area of the recipient using the optical emissions of the device.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a side-by-side comparison of the airway of a human body with and without obstructive sleep apnea, that is useful for understanding the inventive concepts disclosed herein.

FIG. 2 is a perspective view of the obstructive sleep apnea treatment device utilizing optical emitters in accordance with one embodiment of the invention.

FIG. 3a is a bottom side view of the array for use with the obstructive sleep apnea treatment device utilizing optical emitters in accordance with one embodiment of the invention.

FIG. 3b is a perspective side view of the array of the obstructive sleep apnea treatment device utilizing optical emitters in accordance with one embodiment of the invention.

FIG. 4a is a perspective view of the controller of the obstructive sleep apnea treatment device utilizing optical emitters in accordance with one embodiment of the invention.

FIG. 4b is a perspective view of the controller of the obstructive sleep apnea treatment device utilizing optical emitters in accordance with another embodiment of the invention.

FIG. 5a is a schematic block circuit diagram of the controller of the obstructive sleep apnea treatment device utilizing optical emitters in accordance with one embodiment of the invention.

FIG. 5b is a schematic block circuit diagram of the controller of the obstructive sleep apnea treatment device utilizing optical emitters in accordance with another embodiment of the invention.

FIGS. 6a and 6b are diagrams of the modulation input signal and the rectified input signal, respectively, in the controller of FIG. 4a.

FIG. 7 is a perspective view of the obstructive sleep apnea treatment device utilizing optical emitters in operation, and in accordance with one embodiment of the invention.

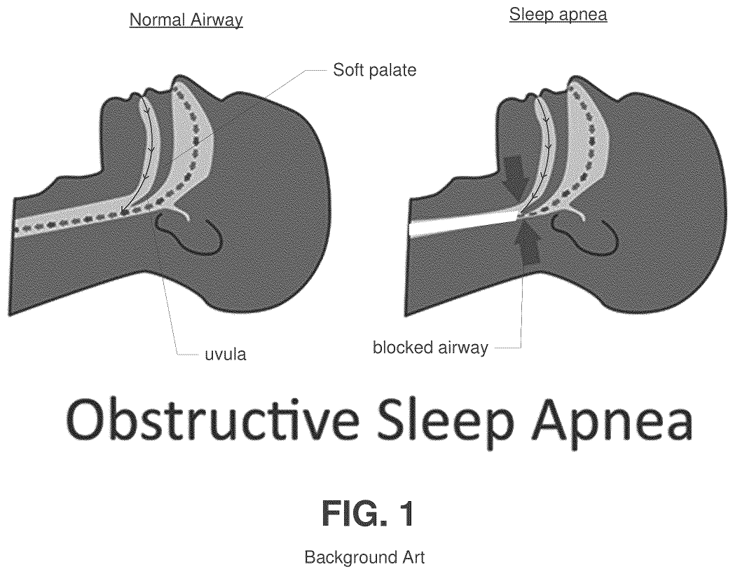

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms.

Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

Through extensive research, testing and development, the inventor has discovered a novel approach for the treatment of Obstructive Sleep Apnea, that can reduce the symptoms and/or presence of the medical condition and sleep disorder. As described below, the treatment can utilize a plurality of optical emitters to supply photonic energy to the target area of the patient at a target wavelength for a specific duration of time.

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of time and/or a component within a tolerance such as, for example, manufacturing tolerances, measurement tolerances, or the like.

As described herein, the “target area” of a person undergoing treatment will be the fat deposits located in the face and neck area including the tongue, soft palate, throat and/or uvula, for example, that can physically compress the airway of the person causing it to shrink, constrict or otherwise collapse during sleep. Of course, other locations along or within the human thorax are contemplated based on the specific treatment to be performed.

As described herein, the term “optical emitter” can include any form of light producing and/or emitting device having any number of different colors across the color spectrum. Several non-limiting examples can include Light Emitting Diodes (“LEDs”), light emitting capacitors, and/or super-luminous light emitting diodes, for example, that are capable of individually and/or jointly creating optical emissions of light/radiation at a target wavelength. In the preferred embodiment, one or more of the optical emitters can output infrared light and visible light having the color red; however, any number of other colors such as yellow and green are also contemplated, for example.

As described herein, the term “target wavelength” can include any wavelength capable of being produced by one or more optical emitters and acting upon the target area of the person. In one preferred embodiment, the target wavelength can be approximately 635 nm.

As described herein, the term “target optical output” describes the measurable light/radiation output of the array that is sufficient to act upon the target area of the person to treat Obstructive Sleep Apnea in the manner described herein. In one preferred embodiment, the target optical output measurable at the array itself shall include an output of red light at the 635 nm target wavelength, at approximately 110,000 Lux.

When the array is positioned at a distance of about 5 cm from the skin/target area of the recipient, the array operating at the target optical output can deliver a target optical delivery of a total luminous flux dose of 30,775 Lux Minutes of 635 nm Light (of the 110,000 produced by the array) to the subcutaneous adipocytes of the treatment area in a treatment time of about 12 minutes. Such a feature being sufficient to reduce the fat within the target area and thus alleviate the onset, symptoms and occurrences of Obstructive Sleep Apnea as described above.

Of course, the distance and time can be adjusted to account for the inherent variances of skin makeup from one person to another. In either instance, upon receiving the target optical delivery produced by the target optical output of the array, a desired energy of 2.88 Joules will be delivered to the person, thus causing the lipid content to be expelled from the adipocytes in the target area without causing cellular damage.

By utilizing Light Emitting Diodes as optical emitters, the present device can output photonic energy across a wide range of wavelengths surrounding the target wavelength of 635 nm. This is because, unlike lasers which are manufactured to produce an identifiable and constant wavelength coherent beam of light, LEDs are generally non-coherent, include a distribution of colors, and are able to generate and safely distribute a greater amount of photonic energy to the human body than lasers.

Therefore, LEDs have a spectrum distribution as opposed to a fixed wavelength that takes into account light output and color. As such, current manufacturing techniques may be unable to provide a plurality of LEDs which can each deliver an exact peak or dominant wavelength at exactly 635 nm on a consistent basis. To this end, when utilizing LEDs, within the below described array, the optical output can include an output spectrum approaching or as close to the target spectrum as possible. Such a feature can be accomplished by individually testing the output of each LED prior to positioning the light within the array, for example.

The below described system and method function to deliver a unique, high-powered red light, which is absorbed by the chromophores of the mitochondria (cytochrome c oxidase), causing the adipocyte to respond as if it was a time of famine. The fat cell's membrane is disrupted, resulting in temporary pores which allow the fat to escape.

When cytochrome c oxidase (“CCO”) absorbs light, it can trigger changes in mitochondrial function, like increased ATP production, which is the basis for therapeutic Photobiomodulation applications. Recipients lose fat permanently through the natural bodily function of lipolysis. The liberated contents of the fat cells are excreted primarily in the recipient's waste, as part of the body's natural detoxification. To this end, the inventive device and method operate at a cellular level and do not generate heat as part of the mechanism of action, but is instead part of an extremely complex, cascading photochemical mechanism.

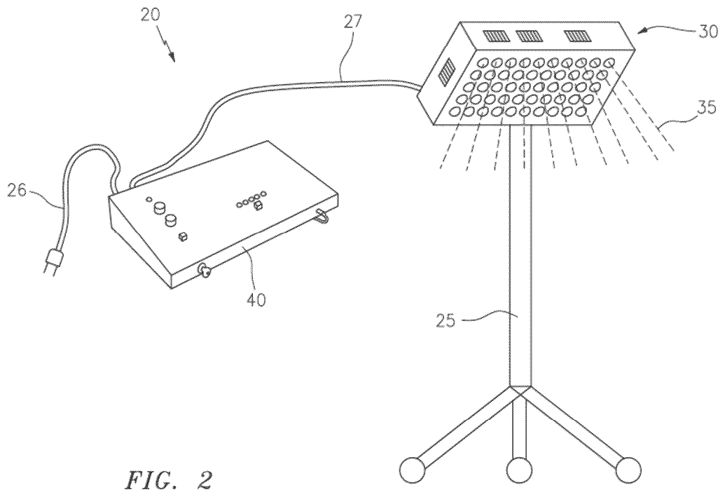

Turning now to the drawings, where identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 2.

FIG. 2 illustrates one embodiment of an Obstructive Sleep Apnea treatment device utilizing optical emitters that is useful for understanding the inventive concepts disclosed herein. As shown, the device 20 can include an array of optical emitters 30 (array) which can operate to produce photonic energy in the form of light/radiation 35 at a target wavelength when instructed by a controller 40. A multi-positional stand 25 can be secured to the array 30 for precise alignment and positioning over a recipient. The controller 40 can include a power cable 26 for mating with a power source, and a second cable 27 can be provided to supply power from the controller 40 to the array 30.

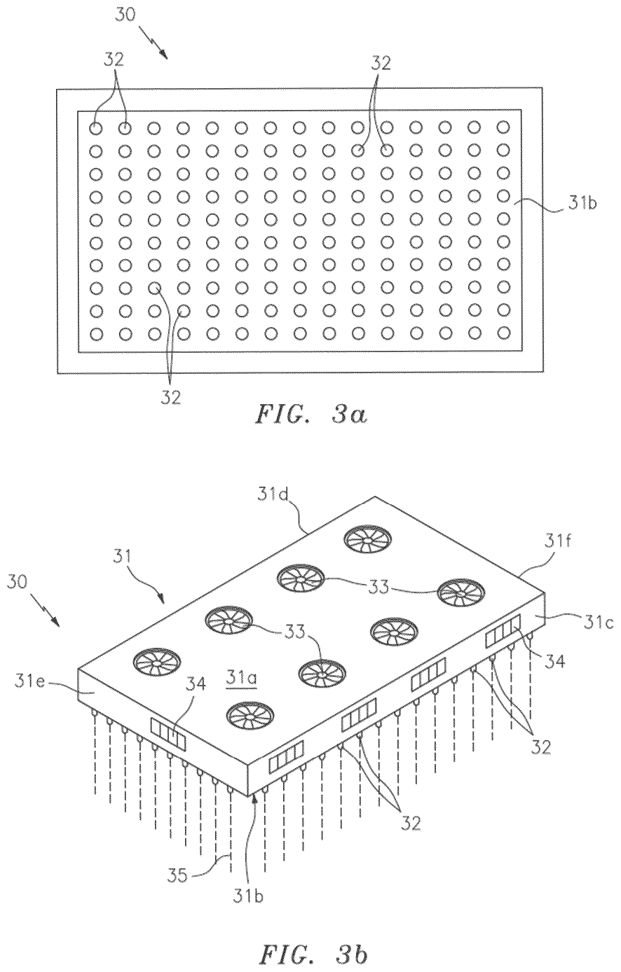

FIGS. 3a and 3b illustrate one embodiment of a suitable array 30. The array can include a main body having a top surface 31a, a bottom surface 31b, a front surface 31c, a back surface 31d and opposing side surfaces 31e and 31f, each forming a generally hollow interior space. A plurality of optical emitters 32 are positioned within the main body 31 so as to allow the emissions therefrom to be directed outward from the bottom surface 31b. Additionally, a plurality of ventilation devices such as fans 33 heat syncs (not shown) and/or air vents 34 can be provided on or within the main body to allow proper air ventilation.

In one preferred embodiment, the main body can be constructed from metal, and the plurality of optical emitters 32 can include 150 2-Watt green LED's that operate at 120V AC power from the cable 27, at the discretion of the controller 40. When power is provided to the array 30, the plurality of emitters 32 can produce the target optical output.

Although described above as including a specific shape, size, construction material, type and number of light emitting sources, this is for illustrative purposes only, as those of skill in the art will recognize that many different combinations and types of optical emitters and/or main body shapes and construction materials can also be utilized to achieve the desired optical output described above. Moreover, although illustrated as protruding out from the main body, the optical emitters 32 can also be flush mounted with the surface of the array itself through manufacturing techniques.

In an alternate embodiment (not illustrated) the main body can comprise a flexible pad containing a plurality of LEDs. In such an embodiment, the plurality of LED's may be operated at lower power levels so as to not require ventilation devices and thus the pad and array may be placed directly on the skin and open mouth of the recipient during a treatment. In such an embodiment, it is contemplated that the light output would be lower than with the fixed array, and thus usage would require a longer treatment time in order to deliver the above noted optimal dose in luminous flux minutes.

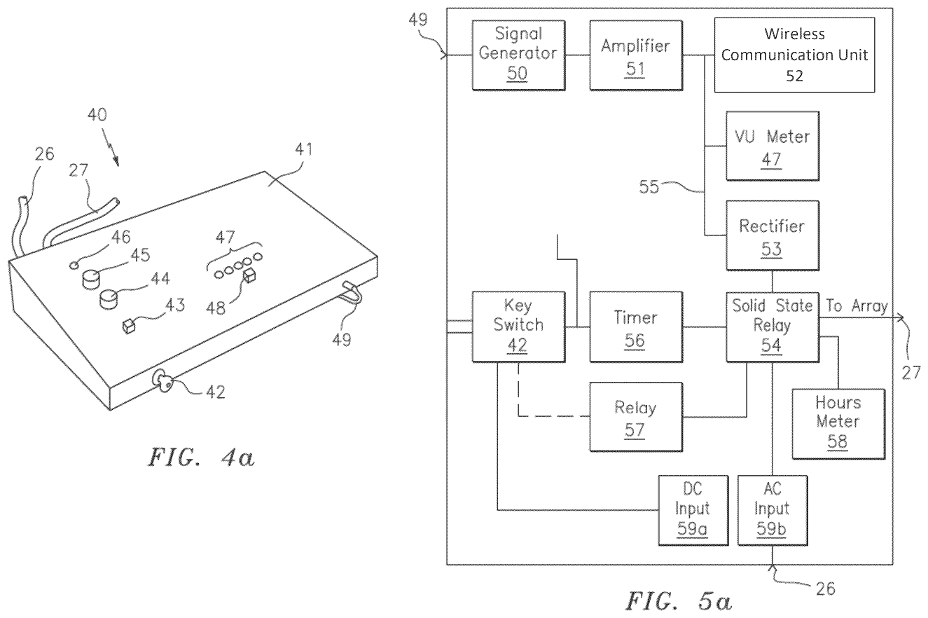

FIG. 4a illustrates an exterior view of one embodiment of the controller 40. As shown, the controller can include a main body 41 having a plurality of user controls and/or interface devices secured thereon. These devices can include, for example, a key switch 42, a stop button 43, a volume selector 44, an exposure time selector 45 an array indicator 46, a modulation signal strength indicator (VU meter) 47, a start button 48 and a signal input jack 49. Of course, any number of other interface devices can also be provided, as necessary or desired to control the functionality of the array.

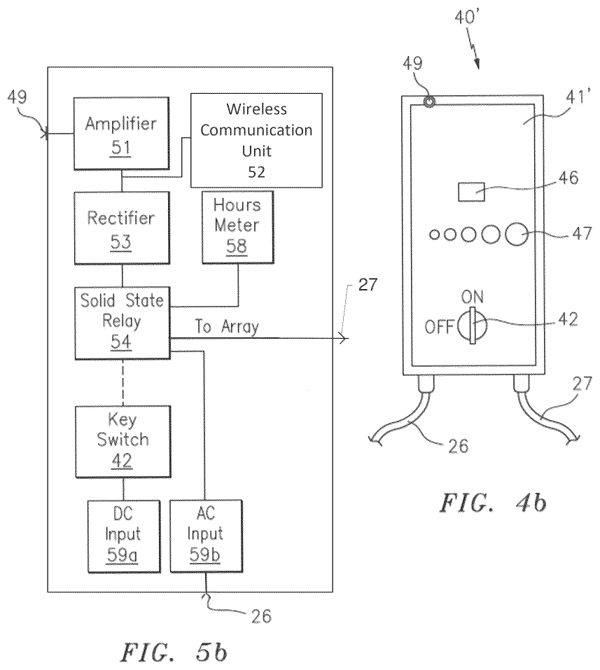

FIG. 5a illustrates one embodiment of a circuitry block diagram of the controller 40 described above. As shown, the internal components can include, for example, a signal generator 50, an amplifier 51, a wireless communication unit 52, a rectifier 53, one or more solid state relays 54, a timer 56, a control switch relay 57, an hours meter 58, an AC power input device 59b, and a DC power module 59a. Each of these components and the user interfaces described above can be communicatively linked via one or more internal cables 55, bus or other suitable device. The above-described diagram is exemplary in nature, as any number of different and/or additional components necessary for operation of the device are also contemplated. For example, any number of LED drivers, power modules and control elements may be provided.

Although described above as including specific features, those of skill in the art will recognize that any means for controlling the operation of the array can be provided herein. To this end, the controller 40 can be manufactured in accordance with any number of known construction methodologies, and one or more of the internal components, although listed as separate elements, can be formed together to form a printed circuit board or other such component, for example, in accordance with known manufacturing processes.

The main body 41 can act to securely position each of the elements 42-59 in secure manner so as to form a single unit which can be operated by a user. The body itself can take any number of distinct shapes and sizes and can be constructed from any number of known materials such as plastic or metal, for example. Of course, other known materials and manufacturing processes are also contemplated.

The key switch 42 can function to provide security against operation of the device by unauthorized persons. To this end, the key switch can interface with a physical key and an internal relay 57 to switch the device between a plurality of operating states. For example, the key can switch the device between: on with internal modulation selected, device off, and device on with external modulation source selected. Although illustrated herein as using a physical key, other access control devices can also be utilized herein. Several non-limiting examples including: key fob, access code (via an integrated keypad), biometric sensors, and the like.

The stop button 43 and key switch can function to immediately cease power distribution to the array 30 when engaged. The volume selector 44 can comprise a switch or knob, for example, which acts to control the output of one or more speakers which may be provided. The exposure time selector 45 can comprise a switch or knob, for example, which can allow a user to specify how long power will be sent to the array during a particular treatment. The array indicator 46 can include a light or other such notification instrument suitable for notifying a user when power is being sent to the array.

The modulation signal strength indicator 47 can act to indicate the presence of an external modulation signal, and to display a visual representation of the signal to a user. The visual representation also indicating the delivery of power to the array 30. The start button 48 functions to initiate the device timer 56 and allows power to be sent to the array 30.

One or more input jacks 49 can be provided to allow the controller 40 to interface with an external device such as a computer/tablet device, radio, television, CD player, DVD player, or MP3 player, for example. These external devices can generate and transmit an audio or sub audio input signal i.e., reference waveform such as music or words, for example, to the controller 40. In one preferred embodiment, the input jack 49 can include a standard ⅛-inch modulation input jack (e.g., mini-phone plug) and/or other forms of input devices such as a USB port, for example.

The input jack 49 can be connected to the internal signal generator 50 such as an audio oscillator, for example, which can act to either generate or pass through the received reference waveform.

The wireless communication unit 52 of the controller can include any number of components capable of sending and/or receiving wireless electronic signals with an externally located device, either directly or over a network. In one preferred embodiment, the communication unit can comprise a Wi-Fi transceiver for communicating wirelessly with a file server over the internet.

Of course, any number of other interface devices can also be provided, as necessary or desired to control the functionality of the array, including digital control using the “Internet of Things” or a wireless device with Bluetooth connectivity to the controller, for example.

In one embodiment, the controller can be connected to a file server over the internet, and the file server can send commands to the controller to operate the device. In another embodiment, a remotely located authorized user may connect to the device controller over the Internet via a browser or application software to receive information from the controller and to send operating commands to the controller. For example, the file server and/or remote user can send commands over the wireless communication unit to activate a time delay relay for a set number of minutes and/or to control other functions of the device such as the array, for example.

In operation, the controller 40 can be connected to a power source such as a conventional 120V AC input source, that can supply power to the internal DC module 49 for powering the controller interfaces. The received AC power can also be modulated and subsequently transferred to the array 30 which can utilize the same to generate an optical output. The controller 40 and/or array 30 can also include any number of fuses to protect the recipient, operator, and device in the event of an electrical problem or excessive current flow through the device components. The flow of electricity (120 VAC) to the array 30 can be controlled by the timer circuit 56, which can interface with the start button 48, stop button 43, the exposure time selector 45 and the array indicator 46.

Together with array positioning relative to the client's body, the controller 40 can vary the fluency of the photonic energy delivered to the skin and underlying tissue. The start button 48 begins the timer circuit 56, which allows power to be sent to the array 30 for conversion into light via the plurality of optical emitters 32. When the array is illuminated, the hours meter 58 advances to record the total operation time of the device.

The controller 40 can execute a method to control the fluency by modulation of the current transmitted to the optical emitters 32 of the array 30. In one embodiment, the current to the array can vary the pulse width (percentage On/Off time) based on the frequency of a reference waveform (i.e., audio input signal) that is internally generated or that is generated by an external device and connected to controller.

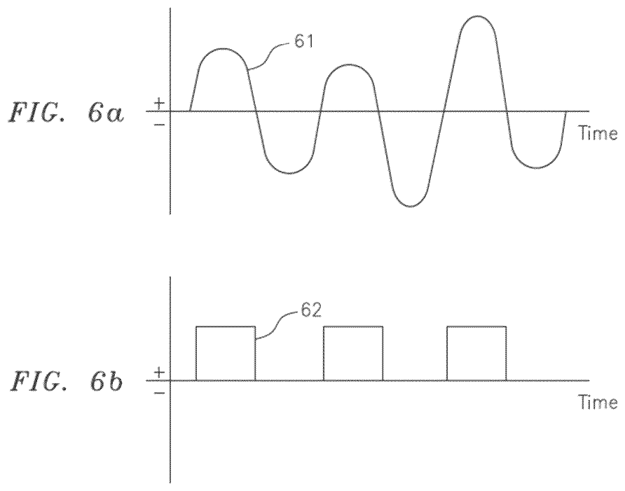

In one embodiment, the reference waveform 61 (see FIG. 6a) can be supplied to the amplifier 51 before being sent to the rectifier 53. The rectifier can function to produce an output voltage representing only the positive portion of the reference waveform. This output voltage 62 (See FIG. 6b) can function to activate the Solid-State Relay (SSR) 54, and the negative portion is shunted to ground. As such, when the SSR is active, 120V AC power can be passed to the array 30 for conversion into light via the plurality of optical emitters 32. Alternatively, when the output voltage 62 is not present, or drops below a predetermined threshold, the SSR can prevent the 120V AC power from passing to the array 30 until the next positive cycle of the reference signal 61. As such, modulation of the array output is accomplished by varying the current and pulse width of the 120V AC voltage supplied to the array 30 from the controller 40.

The VU Meter 47 can be in communication with the signal generator and rectifier in order to provide a visual indicator of the input frequency strength for purposes of adjustment.

Although described above as including the ability to produce a modulated signal, other embodiments are also contemplated. To this end, the device 20 can be operated without modulation, wherein the controller 40 can provide a constant and steady power source to the array 30 for a specific period of time.

Although described above with respect to particular components, those of skill in the art will recognize that the inventive concepts disclosed herein can be accomplished by substituting certain components for other functionally equivalent components, or by reducing the number of components into a more simplified controller.

To this end, FIG. 4b, and 5b, illustrate another embodiment of a controller for operating the array 30.

As shown, controller 40′ can also include a main body 41', having one or more user controls and/or interface devices secured thereon. These devices can also include the above described a key switch 42, an array indicator 46, a modulation signal strength indicator 47, and a signal input jack 49. Of course, any number of other interface devices can also be provided. As shown by the exemplary circuitry block diagram, the controller 40′ can include, for example, the above-described amplifier 51, wireless communication unit 52, rectifier 53, solid state relays 54, hour meter 58, AC power input 59b, and DC power module 59a.

In the present embodiment, the key switch 42 functions to replace the start button and the stop button. In this regard, when the key switch is in the on position, the controller 40′ provides power to the array 30 based on a reference waveform received by the input jack 49, as described above. Conversely, when the key is in the off position, no power is sent to the array, and the DC power to the controller components is disabled. Such a feature can allow the size of the controller to be small in nature, typically between about 14 and 20 inches, so as to enable the device to be easily transported.

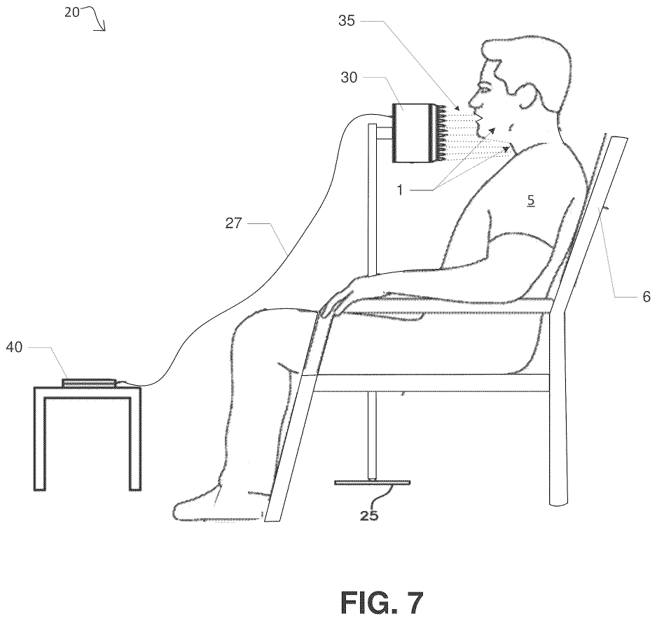

FIG. 7 illustrates one embodiment of the Obstructive Sleep Apnea treatment device 20 utilizing an array of optical emitters 30 to treat a recipient. As shown, the recipient 5 can be positioned in a seated position on a chair 6 or other suitable platform with their mouth open and the stand 25 can be maneuvered to position the array 30 adjacent to the target area 1 which is the mouth, tongue, and neck area of the recipient.

The array can be positioned at any desirable distance from the target area of the recipient and an operator (not shown) or remotely operated device can operate the controller 40 to activate the array. Once activated, the optical emitters of the array can produce the target optical output 35 described above at the target wavelength, for any desirable period of time.

In test results, the optical emitters of the device outputting the target output achieve the target optical delivery of a total luminous flux dose of 30,775 Lux Minutes of 635 nm Light (of the 110,000 produced by the array) to the subcutaneous adipocytes of the treatment area 1 in a treatment time of about 12 minutes when the optical emitters of the array are positioned about 5 cm from the target area 1.

Of course, other embodiments are contemplated wherein the location or output of the device components are different. In either instance, by reducing the fat deposits within the mouth and neck area of the patient, the tongue, soft palate and other areas of the target area are reduced in size and thus do not have the tendence to block the airway of the recipient, thus drastically reducing and/or eliminating instances of obstructive sleep apnea.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Any element in a claim that does not explicitly state “means” for performing a specified function or “step” for performing a specified function should not be interpreted as a “means” or “step” clause as specified in 35 U.S.C. 112. The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A device for treating obstructive sleep apnea, said device comprising:
an output array that includes
a main body having a first surface which may be oriented toward and positioned a first distance from a target area of a recipient to be treated,
an array having a plurality of optical emitters disposed along the main body such that an optical emission from each of the emitters of the array is directed outward from the first surface; and
a controller that is in communication with the array,
said controller including a user interface device for instructing an operation of the array and for transitioning the array between an on-position and an off-position,
wherein the optical emissions are configured to treat an obstructive sleep apnea condition in the recipient.

2. The device of claim 1, wherein each of the plurality of optical emitters comprise, at least one of, a light emitting capacitor, a light emitting diode, and a super-luminous light emitting diode.

3. The device of claim 1, wherein the plurality of optical emitters include 150 2-Watt LEDs having an output wavelength of about 635 nm.

4. The device of claim 1, wherein the controller further includes a key switch access control device, an input jack for receiving a reference waveform from an external device, a power input module, a rectifier and a solid state relay, said rectifier and relay functioning to provide a modulated power output to the array based on the received reference waveform.

5. The device of claim 4, wherein the rectifier and relay function to provide the power output to the array only during a positive portion of the received reference waveform.

6. The device of claim 1, wherein the controller further includes a key switch access control, an internal signal generator for creating a reference waveform, a power input module, a rectifier and a solid state relay, said rectifier and relay functioning to provide a modulated power output to the array based on the created reference waveform.

7. The device of claim 6, wherein the rectifier and relay function to provide the power output to the array only during a positive portion of the created reference waveform.

8. The device of claim 1, wherein the first distance is between 1 and 8 inches, and in the on-position, the array produces green light at approximately 635 nm and at about 383 Lux measurable at a subcutaneous location of the target area of the recipient.

9. The device of claim 1, further comprising:
a stand that is configured to be connected to the main body, said stand including functionality for positioning the main body in an orientation that aligns the array with the target area of the recipient.

10. The device of claim 1, wherein in the on-position the optical output of the array is at a predetermined target optical output that is configured to deliver a target optical delivery to treat obstructive sleep apnea in the recipient when the array is positioned at the first distance from the target area of the recipient.
